(19) United States
(12) Patent Application Publication
(10) Pub. No.: US 2021/0038393 A1
(43) Pub. Date: Feb. 11, 2021

(54) ARTIFICIAL BONE

(71) Applicant: KOLN 3D TECHNOLOGY (MEDICAL) LIMITED, Hong Kong (CN)

(72) Inventors: Wing Fung Edmond YAU, Hong Kong (CN); Sze Yi MAK, Hong Kong (CN); Lung Fung TSE, Hong Kong (CN)

(21) Appl. No.: 16/766,599

(22) PCT Filed: Nov. 29, 2018
(86) PCT No.: PCT/IB2018/059433

§ 371 Date: May 22, 2020

(30) Foreign Application Priority Data
Dec. 1, 2017 (HK) 17112754.2

(51) Int. Cl. A61F 2/28; A61F 2/30; B33Y 80/00

(52) U.S. Cl. CPC A61F 2/2846; A61F 2/30771; B33Y 80/00; A61F 2002/3092; A61F 2002/30331; A61F 2002/30273; B33Y 70/00; A61F 2002/285; A61F 2002/30451; A61F 2002/30405; A61F 2310/00023; A61F 2310/00029; A61F 2002/30784; A61F 2002/30153

(57) ABSTRACT

The utility model provides an artificial bone, comprising bionic bone, supporting pillar and 3D porous scaffold structure; said supporting pillar and said 3D porous scaffold structure are connected to said bionic bone, said 3D porous scaffold structure is set to cover said supporting pillar. Advantageous result of the utility model is: use artificial bone to replace original bone, set supporting pillar and 3D porous scaffold structure in the meantime, supporting pillar gets into coupling end which connected to original bone to work as support and fixing. Set the 3D porous scaffold structure on the periphery of coupling end which connected to original bone, to provide space for the adhesion and growth of bone cells, make the connection between artificial bone and original bone more stable.

### TECHNICAL FIELD

The utility model relates to bionics, in particular to an artificial bone.

### BACKGROUND ART

With the development of science and technology, life span of human is increasing continuously. Nonetheless, the body's function may decline with age or because of disease. Bionic organs are urgently needed to replace native ones.

### SUMMARY OF THE UTILITY MODEL

### Solution to the Problems

In order to solve above existing technical problems, the utility model provides an artificial bone, use the artificial bone to replace original bone; in the meantime, set the supporting pillars and 3D porous scaffold structure to facilitate the connection between artificial bone and original bone and subsequent growth of bone cells.

The utility model solves above existing technical problems, provides an artificial bone, comprising bionic bone, supporting pillars and 3D porous scaffold structure; said supporting pillars and said 3D porous scaffold structure are connected to said bionic bone, said 3D porous scaffold structure is set to cover said supporting pillar.

Further improvements of the utility model are as follows.

Shape of free end of said supporting pillar is changed according to actual need or patient's actual situation.

Said supporting pillar is two columns and the prism between two columns.

Cross section of said prism is rectangle.

Cross section area of said prism is smaller than the cross section area of said column.

Said supporting pillar can get into coupling end which connected to original bone, said 3D porous stent structure is set to cover the coupling end which connected to original bone.

Shape and size of the pore of said 3D porous scaffold structure are changed according to actual need or patient's actual situation.

Said bionic bone, said supporting pillar, and said 3D porous scaffold structure are an integral structure; or, said supporting pillar and said 3D porous scaffold structure are connected to said bionic bone respectively through screw thread; or, said supporting pillar and said 3D porous scaffold structure are connected to said bionic bone respectively through sintering.

### Advantageous Result of Utility Model

In comparison with existing technologies, the advantageous result of the utility model is: use artificial bone to replace original bone, set supporting pillar and 3D porous scaffold structure in the meantime, the supporting pillar gets into coupling end which connected to original bone to work as support and fixing. Set the 3D porous scaffold structure on the periphery of the coupling end which connected to original bone, to provide space for the adhesion and growth of bone cells, and to make the connection between artificial bone and original bone more stable.

### BRIEF DESCRIPTION OF THE DRAWINGS

FIG. 1 is the structural diagram of artificial bone of the utility model;

FIG. 2 is an embodiment diagram of said artificial bone;

FIG. 3 is another embodiment diagram of said artificial bone;

FIG. 4 is the structural diagram of the first embodiment of artificial bone in the utility model;

FIG. 5 is the structural diagram of the second embodiment of artificial bone in the utility model;

FIG. 6 is the structural diagram of the third embodiment of artificial bone in the utility model;

FIG. 7 is the structural diagram of the fourth embodiment of artificial bone in the utility model;

FIG. 8 is the 3D view of the first embodiment of artificial bone in the utility model;

FIG. 9 is the structural diagram of 3D porous scaffold structure of artificial bone in an embodiment in the utility model;

FIG. 10 is the structural diagram of 3D porous scaffold structure of artificial bone in another embodiment in the utility model.

Bionic bone 11; supporting pillar 12; 3D porous scaffold structure 13; original bone 2.

### DETAILED DESCRIPTION OF THE UTILITY MODEL

Below further describe the utility model combining with drawings and embodiment.

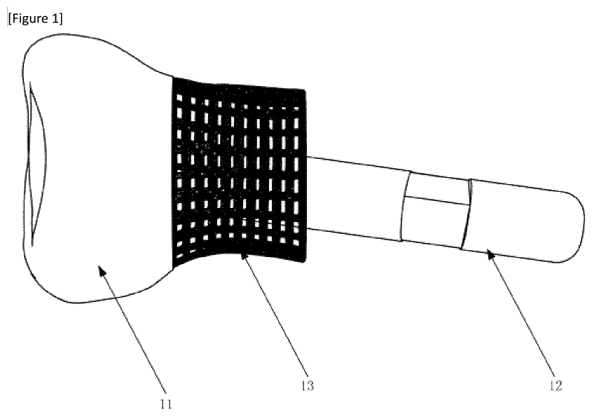

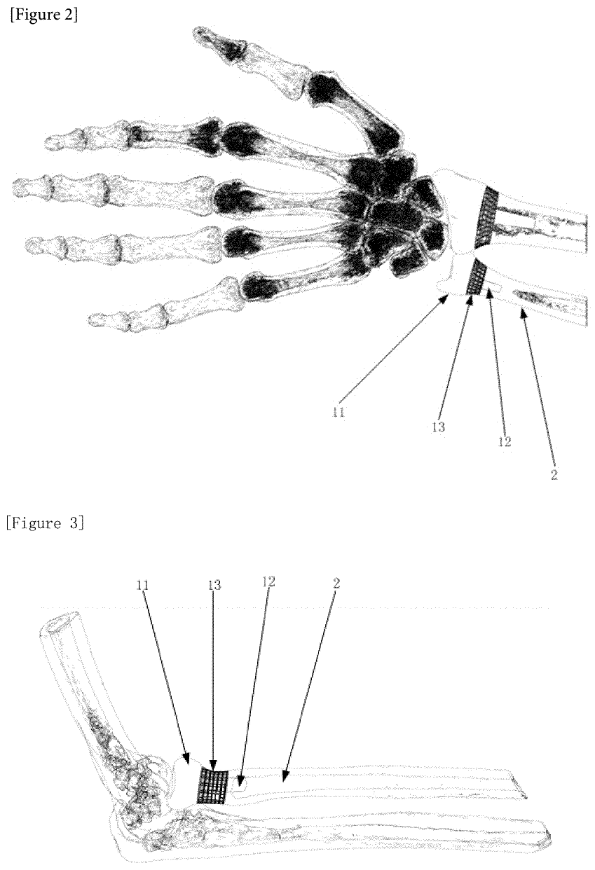

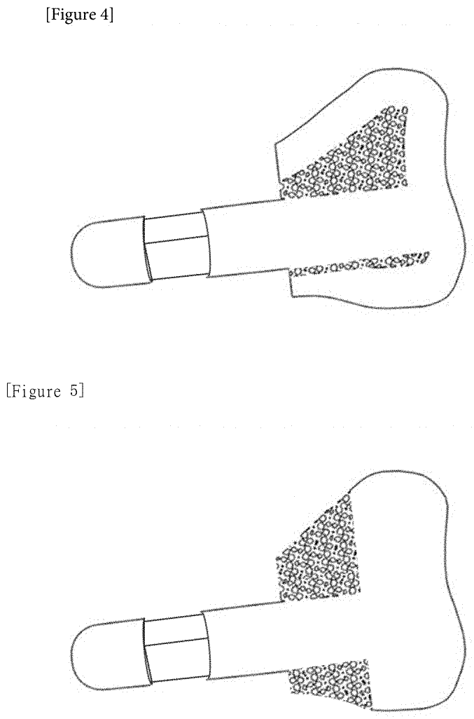

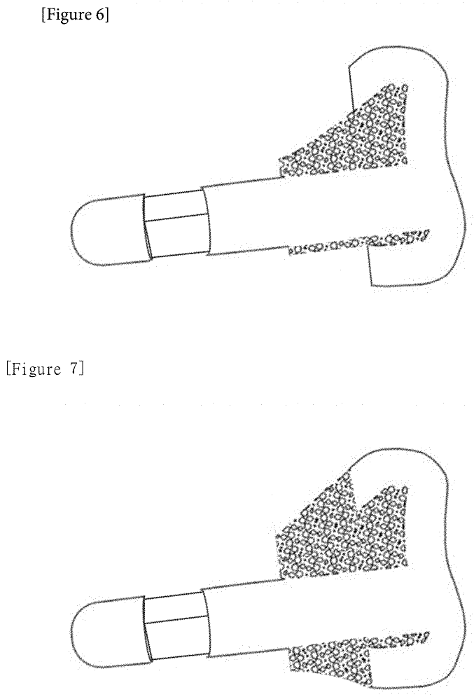

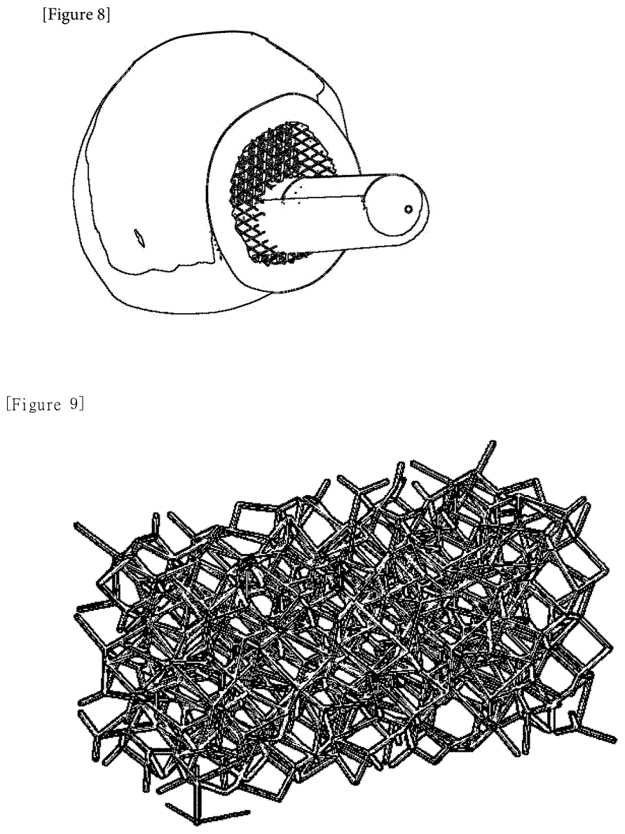

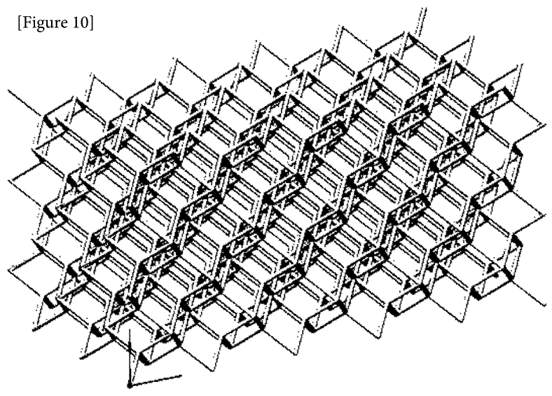

As shown in FIG. 1-3, an artificial bone, comprising bionic bone, supporting pillar and 3D porous scaffold structure; supporting pillar and 3D porous scaffold structure are connected to bionic bone, 3D porous scaffold structure is set to cover the supporting pillar. Use the artificial bone to replace original bone, in the meantime, set the supporting pillar and 3D porous scaffold structure to strengthen the connection between artificial bone and original bone and promote the subsequent growth of bone cells.

Supporting pillar of the utility model is two columns and the prism between two columns, cross section of said prism is rectangle, it can be the fixing part when burnishing, grinding or polishing the bionic bone, also prevent rotation and shift between supporting pillar and original bone. Specifically, cross section area of prism is smaller than the cross section area of the column. Said supporting pillar can get into coupling end which connected to original bone, 3D porous scaffold structure is set to cover the coupling end which connected to original bone.

Shape and size of the pore of said 3D porous scaffold structure in the utility model are changed according to actual need or patient's actual situation.

Bionic bone in the utility model is made according to 3D data of replaced original bone, supporting pillar is made according to inclination of original bone. There are several manufacture methods of artificial bone: in the first method, bionic bone, supporting pillar, and 3D porous scaffold structure are an integral structure. The integral structure is directly printed out by 3D metal printer using cobalt-chromium alloy or titanium alloy. In the second method, bionic bone, supporting pillar, and 3D porous scaffold structure are an integral structure, which is directly printed out by 3D metal printer using titanium alloy, then plated with cobalt-chromium alloy on the surface. In the third method, supporting pillar and 3D porous scaffold structure are connected to bionic bone respectively through screw thread, i.e. use cobalt-chromium alloy to print out the bionic bone by 3D metal printer at first, then use titanium alloy to print out supporting pillar and 3D porous scaffold structure by 3D metal printer, after that assemble the supporting pillar and 3D porous scaffold structure with bionic bone respectively through threaded connection. In the fourth method, supporting pillar and 3D porous scaffold structure are connected to bionic bone by sintering, i.e. use cobalt-chromium alloy to print out the bionic bone by 3D metal printer at first, then use titanium alloy to print out supporting pillar and 3D porous scaffold structure by 3D metal printer, after that sinter the supporting pillar and 3D porous scaffold structure to bionic bone respectively.

Above are the further detailed description of the utility model combining with preferable embodiment, but cannot limit the embodiment of utility model to these only. For the common technician who belongs to the technical field of the utility model, he can make some simple derivations or substitutions on the premise of not departing from the concept of the utility model, it should be deemed as belonging to the protection scope of the utility model.

1. An artificial bone, wherein,
Comprising bionic bone, supporting pillar and 3D porous scaffold structure;
said supporting pillar and said 3D porous scaffold structure are connected to said bionic bone, said 3D porous scaffold structure is set to cover said supporting pillar.

2. The artificial bone of claim 1, wherein, shape of free end of said supporting pillar is changed according to actual need or patient's actual situation.

3. The artificial bone of claim 2, wherein, said supporting pillar is two columns and the prism between two columns.

4. The artificial bone of 3, wherein, cross section of said prism is rectangle.

5. The artificial bone of claim 4, wherein, cross section area of said prism is smaller than the cross section area of said column.

6. The artificial bone of claim 1, wherein, said supporting pillar can get into coupling end which connected to original bone, said 3D porous scaffold structure is set to cover the coupling end which connected to original bone.

7. The artificial bone of claim 1, wherein, shape and size of the hole of said 3D porous scaffold structure are changed according to actual need or patient's actual situation.

8. The artificial bone of claim 1, wherein, said bionic bone and said supporting pillar, said 3D porous scaffold structure are an integral structure; or, said supporting pillar and said 3D porous scaffold structure are connected to said bionic bone through screw thread, respectively; or, said supporting pillar and said 3D porous scaffold structure are connected to said bionic bone by sintering, respectively.
